(19) United States
(12) Patent Application Publication
(10) Pub. No.: US 2024/0207549 A1
(43) Pub. Date: Jun. 27, 2024

(54) APPARATUS FOR DETECTING GAS FLOW AND VENTILATION TREATMENT DEVICE

(71) Applicant: BMC (TIANJIN) MEDICAL CO., LTD., Tianjin (CN)

(72) Inventors: Yawei LIU, Tianjin (CN); Jiuzhong LIU, Tianjin (CN); Shusen YUE, Tianjin (CN); Zhi ZHUANG, Tianjin (CN)

(21) Appl. No.: 18/395,725

(22) Filed: Dec. 25, 2023

(30) Foreign Application Priority Data
Dec. 23, 2022 (CN) 202223470041.6

(51) Int. Cl. A61M 16/00; A61M 16/10

(52) U.S. Cl. CPC A61M 16/0066; A61M 16/0003; A61M 16/107

(57) ABSTRACT

An apparatus for detecting gas flow and a ventilation treatment device. The gas flow channel is provided with at least two detecting holes along a length direction of the gas flow channel, and the two detecting holes are connected to the sensor module by gas paths; the gas resistance module is located in the gas flow channel and located between the two detecting holes; the gas resistance module includes a housing, and an outer wall of the housing attaches to an inner wall of the gas flow channel; an inner wall of the housing surrounds to be a gas passing channel, a filter mesh structure is disposed in the gas passing channel, and the filter mesh structure is connected to the inner wall of the housing; and the filter mesh structure includes an entity part configured for blocking gas and a plurality of ventilation regions separated by the entity part.

### CROSS REFERENCE TO RELATED APPLICATION

This application claims priority to Chinese Patent Application No. 202223470041.6, filed Dec. 23, 2022, the entire disclosure of which is incorporated herein by reference.

### TECHNICAL FIELD

The present application relates to the technical field of medical equipment and more particularly, to an apparatus for detecting gas flow and a ventilation treatment device.

### BACKGROUND

At present, ventilators have been widely used in clinical treatment and hospital emergency rescues. In the process of using the ventilator, it is necessary to collect flow data, and air output of the ventilator is controlled by combining with the actual breathing situation of the patient, to ensure the treatment effect and the comfort level.

In the prior art, a gas resistance module is usually arranged in an airway of the ventilator, and a differential pressure sensor is used to obtain a gas pressure difference at two ends of the gas resistance module, so as to obtain the flow data of the ventilator. When the flow of the ventilator is low, for example, in a child mode, when the existing gas resistance module is used to detect the flow data, there is a problem that an output signal of the gas resistance module is small and a signal-to-noise ratio is low, resulting in that accuracy of the flow data obtained by measuring is low.

### SUMMARY

The present application provides an apparatus for detecting gas flow and a ventilation treatment device, which aim at solving the problem that when the existing gas resistance module is used to measure the flow of the ventilator, the accuracy of the measuring data is low.

In a first aspect, embodiments of the present application disclose an apparatus for detecting gas flow, and the apparatus includes: a gas flow channel, a sensor module and a gas resistance module;
wherein the gas flow channel is provided with at least two detecting holes along a length direction of the gas flow channel, and the two detecting holes are connected to the sensor module by gas paths;
the gas resistance module is located in the gas flow channel and located between the two detecting holes;
the gas resistance module includes a housing, and an outer wall of the housing attaches to an inner wall of the gas flow channel;
an inner wall of the housing surrounds to be a gas passing channel, a filter mesh structure is disposed in the gas passing channel, and the filter mesh structure is connected to the inner wall of the housing; and
the filter mesh structure includes an entity part configured for blocking gas and a plurality of ventilation regions separated by the entity part.

Optionally, both of a cross section of the outer wall of the housing and a cross section of the inner wall of the housing are circular;
the entity part includes a plurality of first gas resistance units arranged in parallel, two ends of each of the plurality of first gas resistance units are connected to the inner wall of the housing, respectively; and
adjacent first gas resistance units are arranged at interval to separate the plurality of ventilation regions.

Optionally, the adjacent first gas resistance units are separated at an equal distance.

Optionally, the entity part further includes at least one second gas resistance unit;
the at least one second gas resistance unit and the plurality of first gas resistance units are crosswise disposed;
two ends of each of the at least one second gas resistance unit are connected to the inner wall of the housing, respectively; and
the plurality of first gas resistance units, the at least one second gas resistance unit and the inner wall of the housing jointly separate the plurality of ventilation regions.

Optionally, the each of the plurality of first gas resistance units and the each of the at least one second gas resistance unit are perpendicular to each other.

Optionally, a cross section of the outer wall of the housing is circular, and a cross section of the inner wall of the housing is rectangular;
the entity part includes a plurality of third gas resistance units arranged in parallel, two ends of each of the plurality of third gas resistance units are connected to the inner wall of the housing, respectively; and
adjacent third gas resistance units are arranged at interval to separate the plurality of ventilation regions.

Optionally, the entity part further includes at least one fourth gas resistance unit;
the at least one fourth gas resistance unit and the plurality of third gas resistance units are crosswise disposed;
two ends of each of the at least one fourth gas resistance unit are connected to the inner wall of the housing, respectively; and
the plurality of third gas resistance units, the at least one fourth gas resistance unit and the inner wall of the housing jointly separate the plurality of ventilation regions.

Optionally, the filter mesh structure includes a punching board, and a circumferential side of the punching board is connected to the inner wall of the housing; and
the punching board is provided with a plurality of hollow holes, each of the plurality of hollow holes corresponds to one ventilation region, and the plurality of hollow holes are separated by the entity part.

Optionally, a cross section of the each of the plurality of hollow holes is circular, and the plurality of hollow holes are evenly distributed on the punching board.

In a second aspect, the embodiments of the present application further disclose a ventilation treatment device, and the ventilation treatment device includes the apparatus for detecting the gas flow stated above.

In the embodiments of the present application, an apparatus for detecting gas flow includes: a gas flow channel, a sensor module and a gas resistance module; wherein the gas flow channel is provided with at least two detecting holes along a length direction of the gas flow channel, and the two detecting holes are connected to the sensor module by gas paths; the gas resistance module is located in the gas flow channel and located between the two detecting holes; the gas resistance module includes a housing, and an outer wall of the housing attaches to an inner wall of the gas flow channel; an inner wall of the housing surrounds to be a gas passing channel, a filter mesh structure is disposed in the gas passing channel, and the filter mesh structure is connected to the inner wall of the housing; and the filter mesh structure includes an entity part configured for blocking gas and a plurality of ventilation regions separated by the entity part. By disposing the filter mesh structure, the length of the contact between the gas flow and the entity part on the overflow cross section is increased, the Reynolds coefficient is reduced, the risk of gas flow turbulence is reduced, the stability of the gas flow is enhanced, the problem of inaccurate data caused by sudden gas flow changes is avoided, thus the measurement accuracy of the flow is improved. Moreover, without changing the size of the gas resistance, the low-flow scenario in the child mode and the high-flow scenario in the adult mode can be compatible, and the use breadth of ventilation treatment device is improved.

### BRIEF DESCRIPTION OF THE DRAWINGS

In order to more clearly illustrate the technical solutions of the embodiments of the present application, the figures that are required in the description of the embodiments of the present application may be briefly introduced below. Obviously, the figures that are described below are merely some embodiments of the present application, and a person skilled in the art can obtain other figures according to these figures without paying creative work.

FIG. 1 shows a first schematic diagram of an assembly structure of a gas resistance module and a gas flow channel in an embodiment of the present application;

FIG. 2 shows a second schematic diagram of an assembly structure of a gas resistance module and a gas flow channel in an embodiment of the present application;

FIG. 3 shows a schematic structural diagram of an apparatus for detecting gas flow in an embodiment of the present application;

FIG. 4 shows a first schematic structural diagram of the gas resistance module in an embodiment of the present application;

FIG. 5 shows a second schematic structural diagram of the gas resistance module in an embodiment of the present application;

FIG. 6 shows a third schematic structural diagram of the gas resistance module in an embodiment of the present application;

FIG. 7 shows a fourth schematic structural diagram of the gas resistance module in an embodiment of the present application; and

FIG. 8 shows a fifth schematic structural diagram of the gas resistance module in an embodiment of the present application.

### DESCRIPTION OF THE REFERENCE NUMBERS

10—gas flow channel; 101—detecting hole; 20—sensor module; 30—gas resistance module; 301—housing; 302—filter mesh structure; 303—entity part; 304—ventilation area; 305—first gas resistance unit; 306—second gas resistance unit; 307—third gas resistance unit; 308—fourth gas resistance unit; 309—hollow hole and 310—punching board.

### DETAILED DESCRIPTION

The technical solutions in the embodiments of the present application may be clearly and completely described below with reference to the drawings of the embodiments of the present application. Apparently, the described embodiments are merely certain embodiments of the present application, rather than all of the embodiments. All of the other embodiments that a person skilled in the art obtains on the basis of the embodiments of the present application without paying creative work fall within the protection scope of the present application.

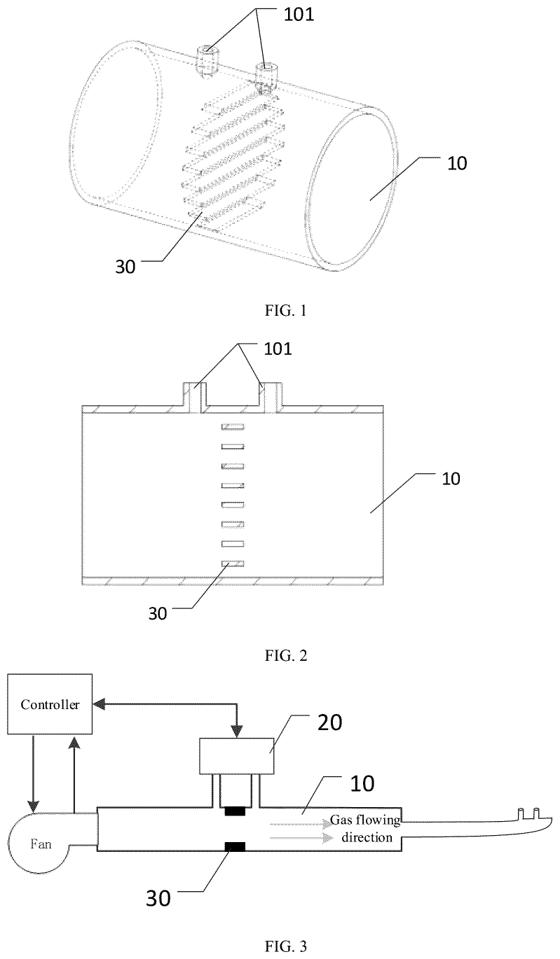

Referring to FIG. 1 to FIG. 8, an apparatus for detecting gas flow is disclosed by the embodiments of the present application, the apparatus includes: a gas flow channel 10, a sensor module 20 and a gas resistance module 30; the gas flow channel 10 is provided with at least two detecting holes 101 along a length direction of the gas flow channel, and the two detecting holes 101 are connected to the sensor module 20 by gas paths; the gas resistance module 30 is located in the gas flow channel 10 and located between the two detecting holes 101; the gas resistance module 30 includes a housing 301, and an outer wall of the housing 301 attaches to an inner wall of the gas flow channel 10; an inner wall of the housing 301 surrounds to be a gas passing channel, a filter mesh structure 302 is disposed in the gas passing channel, and the filter mesh structure 302 is connected to the inner wall of the housing 301; and the filter mesh structure 302 includes an entity part 303 configured for blocking gas and a plurality of ventilation regions 304 separated by the entity part 303.

Specifically, two ends of the gas flow channel 10 are connected to a fan outlet of the ventilator and a patient end, respectively, to transmit the pressurized gas generated by the ventilator to the patient end. The gas flow channel 10 is provided with at least two detecting holes 101 along the length direction of the gas flow channel, the two detecting holes 101 are located upstream and downstream of the gas flow, respectively. The gas resistance module 30 is located in the gas flow channel 10 and located between the two detecting holes 101. The gas resistance module 30 and the gas flow channel 10 may be of a separating structure, and the gas resistance module 30 may be separately placed in the gas flow channel 10. The gas resistance module 30 and the gas flow channel 10 may also be of an integrated structure, and the gas resistance module 30 may be fixed in the gas flow channel 10.

When the gas flow channel 10 is ventilated, the gas flow passes through the gas resistance module 30, and a pressure difference is generated between the detecting holes 101 at the two ends of the gas resistance module 30. The sensor module 20 is connected to the two detecting holes 101 by gas paths, respectively, and the pressure difference between the two detecting holes 101 can be obtained, and then the flow of the ventilator can be measured.

The gas resistance module 30 includes a housing 301, and an outer wall of the housing 301 attaches to an inner wall of the gas flow channel 10. For example, the cross section of the inner wall of the gas flow channel 10 is circular, correspondingly, the cross section of the outer wall of the housing 301 is also circular. An inner wall of the housing 301 surrounds to be a gas passing channel, the gas passing channel is coaxially disposed with the gas flow channel 10. A filter mesh structure 302 is disposed in the gas passing channel, and the filter mesh structure 302 is connected to the inner wall of the housing 301. The filter mesh structure 302 includes an entity part 303 configured for blocking gas and a plurality of ventilation regions 304 separated by the entity part 303. The gas flow can pass through the gas resistance module 30 through the ventilation region 304, and the entity part 303 and the inner wall of the housing 301 may be of a separating structure or may also be of an integrated structure.

Gas resistance refers to the ability of the solid medium to hinder the flowing of the gas flow. When the pipe diameter of the outer wall of the housing 301 is constant, the larger the area of the solid medium is, the smaller the ventilation area is, and the larger the corresponding gas resistance is, on the contrary, the smaller the gas resistance is.

In fluid mechanics, the Reynolds coefficient Rₑ is used to reflect the change of the gas flow:

$${R}_{e} = \frac{v d}{μ} \qquad ( 1 )$$

The Reynolds coefficient of the circular tube is shown in formula (1), in which v, d and μ are the flow velocity, the pipe diameter and the kinematic viscosity of the fluid, respectively. The experimental results show that the critical Reynolds coefficient Rₑ of the fluid flowing in the circular tube transferring from turbulence to laminar flow is about 2300, which is quite stable. The critical Reynolds coefficient of the fluid flowing in the circular tube transferring from the laminar flow to the turbulence is related to the experimental environment, which generally changes between 4000 and 20000. Therefore, Rₑ=2300 is taken as the basis for distinguishing the flow pattern. When Rₑ<2300, the flow pattern of the fluid is laminar flow. When Rₑ>2300, the flow pattern of the fluid is turbulence.

For the gas resistance of the circular tube structure in the prior art, the area enclosed by the cylindrical tube wall is the ventilation region 304 for flowing gas, and the tube wall is used for blocking the gas. According to the formula (1), the Reynolds coefficient can be reduced by reducing the flow velocity v or the pipe diameter d, but in this way, the output flow of the ventilator may be reduced or the load of the fan may be increased, which cannot meet requirement of the usage scenario of high flow, it is easy to cause the damage of the fan, and it is not conducive to improving the use breadth and durability of the ventilator.

For the flowing in a non-circular pipe, the hydraulic radius R can be used as the characteristic length d in the Reynolds coefficient, as shown in formula (2).

$$R = \frac{A}{x} \qquad ( 2 )$$

Where A is the area of the overflow cross section, x is a length that the entity part 303 on the overflow cross section and the inner wall of the housing 301 contact with the fluid, and x is called wetted perimeter. In combination with formula (1), the corresponding Reynolds coefficient is shown in formula (3).

$${R}_{e} = \frac{v A}{μ x} \qquad ( 3 )$$

In the actual gas resistance design, the Reynolds coefficient Rₑ can be reduced, so that the gas flow is in a laminar flow state, and the gas flow measurement accuracy can be improved. The kinematic viscosity of air under a standard condition is μ=14.8*10⁻⁶ m²/s.

If the gas resistance module 30 is a non-circular gas resistance, according to the formula (3), the Reynolds coefficient can be reduced by reducing the flow rate v, reducing the area A of the cross section or increasing the wetted perimeter x, while reducing the flow rate and reducing the area of the cross section may bring negative effects.

Therefore, in the embodiments of the present invention, under the premise of ensuring that the gas resistance remains unchanged, the shape of the gas resistance is changed, and the ventilation cross section is changed from a circular structure to a rectangular structure or a filter mesh structure 302. By increasing the wetted perimeter x, the Reynolds coefficient is reduced, the risk of gas flow turbulence can be reduced, the stability of the gas flow can be enhanced, and the problem of inaccurate data caused by sudden gas flow changes can be avoided, thereby the measurement accuracy of the flow is improved.

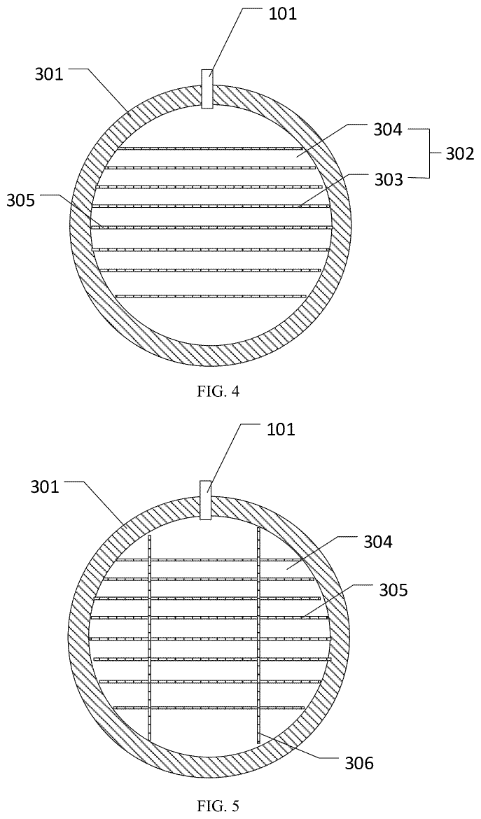

Optionally, referring to FIG. 4, both of a cross section of the outer wall of the housing 301 and a cross section of the inner wall of the housing 301 are circular; the entity part 303 includes a plurality of first gas resistance units 305 arranged in parallel, two ends of each of the plurality of first gas resistance units 305 are connected to the inner wall of the housing 301, respectively; and adjacent first gas resistance units 305 are arranged at interval to separate the plurality of ventilation regions 304.

Specifically, in an embodiment of the present invention, in the gas resistance module 30, both of the cross section of the outer wall of the housing 301 and the cross section of the inner wall of the housing 301 are circular to form a cylindrical gas detection channel, and the filter mesh structure 302 is disposed in the gas detection channel. The entity part 303 of the filter mesh structure 302 includes the plurality of first gas resistance units 305 arranged in parallel. The plurality of first gas resistance units 305 are arranged in parallel on the cross section of the housing 301. The first gas resistance unit 305 may be a rectangular anti-aeration plate. A length direction of the anti-aeration plate is parallel to the radial direction of the cross section of the housing 301, and a width of the anti-aeration plate is not greater than the distance between the two detecting holes 101. The two ends of each of the plurality of first gas resistance units 305 are connected to the inner wall of the housing 301, respectively, which can be specifically fixed by using a method such as bonding, clamping and welding and so on. The housing 301 and the first gas resistance unit 305 can also be made by using a method of integrated molding, and the structural strength is better. The quantity and the thickness of the first gas resistance units 305 can be selected according to the size of the housing 301, the design size of gas resistance and the ventilation area.

Adjacent first gas resistance units 305 are arranged at interval to separate the plurality of ventilation regions 304. When the gas flow passes through the filter mesh structure 302, there is a contact surface between the first gas resistance unit 305 and the gas flow, thus the wetted perimeter can be increased, and the Reynolds coefficient may be reduced to reduce the risk of gas flow turbulence, which enhances the stability of the gas flow, and avoids the problem of inaccurate data caused by sudden gas flow changes, thereby improving the measurement accuracy of the flow.

Optionally, referring to FIG. 4, the adjacent first gas resistance units 305 are separated at an equal distance.

Specifically, the adjacent first gas resistance units 305 are separated at an equal distance, so that the plurality of ventilation regions 304 with a consistent height are formed by separating. The height direction is the vertical connection direction of the adjacent first gas resistance units 305. When the gas flow passes through the plurality of ventilation regions 304 with the consistent height, the uniformity is better, and the flow measurement accuracy is further improved.

Optionally, referring to FIG. 5, the entity part 303 further includes at least one second gas resistance unit 306; the at least one second gas resistance unit 306 and the plurality of first gas resistance units 305 are crosswise disposed; two ends of each of the at least one second gas resistance unit 306 are connected to the inner wall of the housing 301, respectively; and the plurality of first gas resistance units 305, the at least one second gas resistance unit 306 and the inner wall of the housing 301 jointly separate the plurality of ventilation regions 304.

Specifically, the second gas resistance unit 306 and the plurality of first gas resistance units 305 are crosswise disposed to form a grid structure, which can reinforce the plurality of first gas resistance units 305 to avoid the problem of vibration caused by gas flow impact. At the same time, under the premise of ensuring that ventilation areas remain unchanged, the area of the windward surface of a single gas resistance unit can be reduced, and the quantity of the gas resistance units can be increased, that is, the quantity of the windward surfaces of the gas resistance units can be increased, the risk of gas flow turbulence can also be reduced to enhance the stability of the gas flow.

The methods for connecting the second gas resistance unit 306 to the first gas resistance unit 305 and the inner wall of the housing 301 may be bonding, clamping, welding, etc., and the second gas resistance unit 306, the first gas resistance unit 305 and the inner wall of the housing 301 can also be made by using a method of integrated molding, the structural strength is better.

Optionally, referring to FIG. 5, the each of the plurality of first gas resistance units 305 and the each of the at least one second gas resistance unit 306 are perpendicular to each other.

Specifically, in the plurality of ventilation regions 304 separated by the first gas resistance unit 305 and the second gas resistance unit 306 that are perpendicular to each other, the quantity of ventilation regions 304 with the equal area is more, which is beneficial to improve the stability of passing the gas flow.

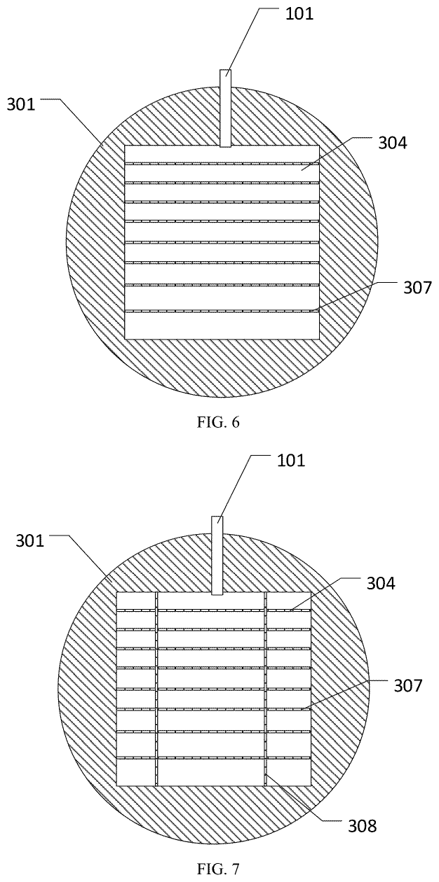

Optionally, referring to FIG. 6, a cross section of the outer wall of the housing 301 is circular, and a cross section of the inner wall of the housing 301 is rectangular; the entity part 303 includes a plurality of third gas resistance units 307 arranged in parallel, two ends of each of the plurality of third gas resistance units 307 are connected to the inner wall of the housing 301, respectively; and adjacent third gas resistance units 307 are arranged at interval to separate the plurality of ventilation regions 304.

Specifically, in another embodiment of the present invention, in the gas resistance module 30, a cross section of the outer wall of the housing 301 is circular, and a cross section of the inner wall of the housing 301 is rectangular to form a rectangular gas detection channel. The filter mesh structure 302 is disposed in the gas detection channel. Under the premise of the same ventilation area, the rectangular cross section has a larger perimeter than the circular cross section, thus the wetted perimeter can be increased and the Reynolds coefficient can be reduced.

The entity part 303 of the filter mesh structure 302 includes the plurality of third gas resistance units 307 arranged in parallel, the plurality of third gas resistance units 307 are arranged in parallel on the cross section of the housing 301, and may be specifically arranged along the length or the width direction of the rectangular cross section, or may also be arranged along the diagonal direction of the rectangular cross section. The third gas resistance unit 307 may be a rectangular anti-aeration plate. The length direction of the anti-aeration plate is parallel to the length or width direction of the rectangular cross section, and may also be parallel to the diagonal direction of the rectangular cross section. The width of the anti-aeration plate is not greater than the distance between the two detecting holes 101. The two ends of the third gas resistance unit 307 are connected to the inner wall of the housing 301, respectively, which can be specifically fixed by using a method such as bonding, clamping and welding and so on. The housing 301 and the third gas resistance unit 307 can also be made by using a method of integrated molding, and the structural strength is better. The quantity and the thickness of the third gas resistance units 307 can be selected according to the size of the housing 301, the design size of gas resistance and the ventilation area.

The adjacent third gas resistance units 307 are arranged at interval to separate the plurality of ventilation regions 304. When the gas flow passes through the filter mesh structure 302, there is a contact surface between the third gas resistance unit 307 and the gas flow, thus the wetted perimeter can be increased, and the Reynolds coefficient may be reduced to reduce the risk of gas flow turbulence, which enhances the stability of the gas flow, and avoids the problem of inaccurate data caused by sudden gas flow changes, thereby improving the measurement accuracy of the flow.

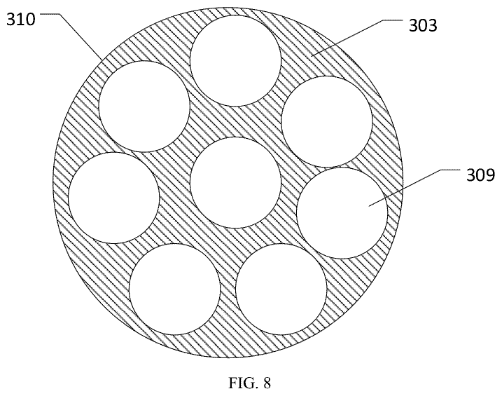

Optionally, referring to FIG. 7, the entity part 303 further includes at least one fourth gas resistance unit 308; the at least one fourth gas resistance unit 308 and the plurality of third gas resistance units 307 are crosswise disposed; two ends of each of the at least one fourth gas resistance unit 308 are connected to the inner wall of the housing 301, respectively; and the plurality of third gas resistance units 307, the at least one fourth gas resistance unit 308 and the inner wall of the housing 301 jointly separate the plurality of ventilation regions 304.

Specifically, the fourth gas resistance unit 308 and the plurality of third gas resistance units 307 are crosswise disposed to form a grid structure, which can reinforce the plurality of third gas resistance units 307 to avoid the problem of vibration caused by gas flow impact. At the same time, under the premise of ensuring that the ventilation areas remain unchanged, the area of the windward surface of a single gas resistance unit can be reduced, and the quantity of the gas resistance units can be increased, that is, the quantity of the windward surfaces of the gas resistance units can be increased, the risk of gas flow turbulence can also be reduced to enhance the stability of the gas flow.

The methods for connecting the fourth gas resistance unit 308 to the third gas resistance unit 307 and the inner wall of the housing 301 may be bonding, clamping, welding, etc., and that the fourth gas resistance unit 308, the third gas resistance unit 307 and the inner wall of the housing 301 can also be made by using a method of integrated molding, the structural strength is better.

In addition, the third gas resistance units 307 and the fourth gas resistance unit 308 are preferably arranged vertically. In the plurality of ventilation regions 304 that are separated, the quantity of ventilation regions 304 with the equal area is more, which is beneficial to improve the stability of the gas flow passing.

Optionally, referring to FIG. 8, the filter mesh structure 302 includes a punching board 310, and a circumferential side of the punching board 310 is connected to the inner wall of the housing 301; and the punching board 310 is provided with a plurality of hollow holes 309, each of the plurality of hollow holes 309 corresponds to one ventilation region 304, and the plurality of hollow holes 309 are separated by the entity part 303.

Specifically, in yet another embodiment of the present invention, the filter mesh structure 302 is composed of the punching board 310. The shape of the punching board 310 can be circular, rectangular, etc. The circumferential side of the punching board 310 is connected to the inner wall of the housing 301. The specific connection methods can be bonding, welding, clamping, etc. The punching board 310 can also be formed in the housing 301 by using an integrated molding process. The punching board 310 is provided with a plurality of hollow holes 309. The shape of each of the hollow holes 309 can be circular, rectangular, polygonal, etc. The quantity of the hollow holes 309 can be selected according to the actual needs. Each hollow hole 309 corresponds to one ventilation region 304, and the region on the punching board 310 except the hollow hole 309 is the entity part 303, and the plurality of hollow holes 309 are separated by the entity part 303. During ventilation, the hole circumference of each hollow hole 309 is in contact with the gas flow, which can increase the wetted perimeter, and reduce the Reynolds coefficient, to reduce the risk of gas flow turbulence, enhance the stability of the gas flow, and avoid the problem of inaccurate data caused by sudden gas flow changes, thereby improving the measurement accuracy of the flow.

Optionally, referring to FIG. 8, a cross section of each of the plurality of hollow holes 309 is circular, and the plurality of hollow holes 309 are evenly distributed on the punching board 310.

Specifically, the cross section of each of the hollow holes 309 is circular, and the circular hollow hole is used to make the gas flow pass more smoothly. The hollow holes 309 are evenly distributed on the punching board 310, so that the gas flow can be evenly separated by the plurality of hollow holes 309, and the uniformity is better. It is beneficial to improving the stability of the gas flow and further improving the flow measurement accuracy.

The embodiments of the present application further disclose a ventilation treatment device, which includes the apparatus for detecting the gas flow stated above.

Specifically, the ventilation treatment device may include ventilators, high-flow humidified oxygen therapy devices and other devices. The apparatus for detecting gas flow is placed in the ventilation pipeline of the ventilator or the high-flow humidified oxygen therapy device, to achieve flow detection. In addition, the ventilation treatment device can also include a controller. The controller is used to control the air output and the working time, etc. of the ventilation treatment device. The controller may be an electronic device or may also be a component in the electronic device, such as an integrated circuit or a chip. The electronic device may be a terminal or other devices besides the terminal. For example, electronic devices may be mobile phones, tablets, laptops, handheld computers, mobile internet devices, robots, wearable devices, etc., which is not limited by the embodiments of the present application.

In the embodiments of the present application, the ventilation treatment device uses the apparatus for detecting the gas flow stated above. The apparatus for detecting the gas flow includes a gas flow channel 10, a sensor module 20 and a gas resistance module 30; the gas flow channel 10 is provided with at least two detecting holes 101 along a length direction of the gas flow channel, and the two detecting holes 101 are connected to the sensor module 20 by gas paths; the gas resistance module 30 is located in the gas flow channel 10 and located between the two detecting holes; the gas resistance module 30 includes a housing 301, and an outer wall of the housing 301 attaches to an inner wall of the gas flow channel 10; an inner wall of the housing 301 surrounds to be a gas passing channel, a filter mesh structure 302 is disposed in the gas passing channel, and the filter mesh structure 302 is connected to the inner wall of the housing 301; and the filter mesh structure 302 includes an entity part 303 configured for blocking gas and a plurality of ventilation regions 304 separated by the entity part 303. By disposing the filter mesh structure 302, the length of the contact between the gas flow and the entity part 303 on the overflow cross section is increased, the Reynolds coefficient is reduced, the risk of gas flow turbulence is reduced, the stability of the gas flow is enhanced, and the problem of inaccurate data caused by sudden gas flow changes is avoided, thus the measurement accuracy of the flow is improved. Moreover, without changing the size of the gas resistance, the low-flow scenario in the child mode and the high-flow scenario in the adult mode can be compatible, and the use breadth of ventilation treatment device is improved.

It should be noted that, the term “includes”, “comprises” or any other variation thereof herein is intended to cover non-exclusive inclusion, so that a process, method, goods or device that includes a set of elements includes not only those elements but also other elements not explicitly listed, or include elements inherent to the process, method, object, or device. In the absence of further restrictions, the limited element stated by “including a . . . ” does not preclude the existence of another identical element in the process, method, goods or device that includes the element.

The embodiments of the present application are described above in combination with the drawings. However, the present application is not limited to the above specific embodiments, which are only indicative rather than restrictive. Under the inspiration of the present application, a person skilled in the art can also make many forms without deviating from the scope protected by the purposes of the present application and the claims are covered by the present application.

1. An apparatus for detecting gas flow, comprising:
a gas flow channel, a sensor module and a gas resistance module;
wherein the gas flow channel is provided with at least two detecting holes along a length direction of the gas flow channel, and two detecting holes are connected to the sensor module by gas paths;
wherein the gas resistance module is located in the gas flow channel and located between the two detecting holes;
wherein the gas resistance module comprises a housing, and an outer wall of the housing attaches to an inner wall of the gas flow channel;
wherein an inner wall of the housing surrounds to be a gas passing channel, a filter mesh structure is disposed in the gas passing channel, and the filter mesh structure is connected to the inner wall of the housing; and
wherein the filter mesh structure comprises an entity part configured for blocking gas and a plurality of ventilation regions separated by the entity part.

2. The apparatus for detecting the gas flow according to claim 1, wherein both of a cross section of the outer wall of the housing and a cross section of the inner wall of the housing are circular;
wherein the entity part comprises a plurality of first gas resistance units arranged in parallel, two ends of each of the plurality of first gas resistance units are connected to the inner wall of the housing, respectively; and
wherein adjacent first gas resistance units are arranged at interval to separate the plurality of ventilation regions.

3. The apparatus for detecting the gas flow according to claim 2, wherein the adjacent first gas resistance units are separated at an equal distance.

4. The apparatus for detecting the gas flow according to claim 2, wherein the entity part further comprises at least one second gas resistance unit;
wherein the at least one second gas resistance unit and the plurality of first gas resistance units are crosswise disposed;
wherein two ends of each of the at least one second gas resistance unit are connected to the inner wall of the housing, respectively; and
wherein the plurality of first gas resistance units, the at least one second gas resistance unit and the inner wall of the housing jointly separate the plurality of ventilation regions.

5. The apparatus for detecting the gas flow according to claim 4, wherein the each of the plurality of first gas resistance units and the each of the at least one second gas resistance unit are perpendicular to each other.

6. The apparatus for detecting the gas flow according to claim 1, wherein a cross section of the outer wall of the housing is circular, and a cross section of the inner wall of the housing is rectangular;
wherein the entity part comprises a plurality of third gas resistance units arranged in parallel, two ends of each of the plurality of third gas resistance units are connected to the inner wall of the housing, respectively; and
wherein adjacent third gas resistance units are arranged at interval to separate the plurality of ventilation regions.

7. The apparatus for detecting the gas flow according to claim 6, wherein the entity part further comprises at least one fourth gas resistance unit;
wherein the at least one fourth gas resistance unit and the plurality of third gas resistance units are crosswise disposed;
wherein two ends of each of the at least one fourth gas resistance unit are connected to the inner wall of the housing, respectively; and
wherein the plurality of third gas resistance units, the at least one fourth gas resistance unit and the inner wall of the housing jointly separate the plurality of ventilation regions.

8. The apparatus for detecting the gas flow according to claim 1, wherein the filter mesh structure comprises a punching board, and a circumferential side of the punching board is connected to the inner wall of the housing; and
wherein the punching board is provided with a plurality of hollow holes, each of the plurality of hollow holes corresponds to one ventilation region, and the plurality of hollow holes are separated by the entity part.

9. The apparatus for detecting the gas flow according to claim 8, wherein a cross section of the each of the plurality of hollow holes is circular, and the plurality of hollow holes are evenly distributed on the punching board.

10. A ventilation treatment device, comprising the apparatus for detecting the gas flow according to claim 1.

11. The ventilation treatment device according to claim 10, wherein both of a cross section of the outer wall of the housing and a cross section of the inner wall of the housing are circular;
wherein the entity part comprises a plurality of first gas resistance units arranged in parallel, two ends of each of the plurality of first gas resistance units are connected to the inner wall of the housing, respectively; and
wherein adjacent first gas resistance units are arranged at interval to separate the plurality of ventilation regions.

12. The ventilation treatment device according to claim 11, wherein the adjacent first gas resistance units are separated at an equal distance.

13. The ventilation treatment device according to claim 11, wherein the entity part further comprises at least one second gas resistance unit;
wherein the at least one second gas resistance unit and the plurality of first gas resistance units are crosswise disposed;
wherein two ends of each of the at least one second gas resistance unit are connected to the inner wall of the housing, respectively; and
wherein the plurality of first gas resistance units, the at least one second gas resistance unit and the inner wall of the housing jointly separate the plurality of ventilation regions.

14. The ventilation treatment device according to claim 13, wherein the each of the plurality of first gas resistance units and the each of the at least one second gas resistance unit are perpendicular to each other.

15. The ventilation treatment device according to claim 10, wherein a cross section of the outer wall of the housing is circular, and a cross section of the inner wall of the housing is rectangular;
wherein the entity part comprises a plurality of third gas resistance units arranged in parallel, two ends of each of the plurality of third gas resistance units are connected to the inner wall of the housing, respectively; and
wherein adjacent third gas resistance units are arranged at interval to separate the plurality of ventilation regions.

16. The ventilation treatment device according to claim 15, wherein the entity part further comprises at least one fourth gas resistance unit;
wherein the at least one fourth gas resistance unit and the plurality of third gas resistance units are crosswise disposed;
wherein two ends of each of the at least one fourth gas resistance unit are connected to the inner wall of the housing, respectively; and
wherein the plurality of third gas resistance units, the at least one fourth gas resistance unit and the inner wall of the housing jointly separate the plurality of ventilation regions.

17. The ventilation treatment device according to claim 10, wherein the filter mesh structure comprises a punching board, and a circumferential side of the punching board is connected to the inner wall of the housing; and
wherein the punching board is provided with a plurality of hollow holes, each of the plurality of hollow holes corresponds to one ventilation region, and the plurality of hollow holes are separated by the entity part.

18. The ventilation treatment device according to claim 17, wherein a cross section of the each of the plurality of hollow holes is circular, and the plurality of hollow holes are evenly distributed on the punching board.
